(19) United States
(12) Patent Application Publication
(10) Pub. No.: US 2011/0059537 A1
(43) Pub. Date: Mar. 10, 2011

(54) METHOD FOR ESTIMATING RISK OF ACUTE KIDNEY INJURY

(75) Inventors: Orfeas Liangos, Brighton, MA (US); Francesco Addabbo, Valhalla, NY (US); Michael Goligorsky, Valhalla, NY (US); Bertrand L. Jaber, South Boston, MA (US)

(73) Assignees: Caritas St. Elizabeth's Medical Center of Boston, Inc., Boston, MA (US); New York Medical College, Valhalla, NY (US)

(21) Appl. No.: 12/677,393

(22) PCT Filed: Sep. 18, 2008
(86) PCT No.: PCT/US08/10860

§ 371 Date: Nov. 22, 2010

Related U.S. Application Data
(60) Provisional application No. 60/994,587, filed on Sep. 20, 2007.

(51) Int. Cl. G01N 33/53

(52) U.S. Cl. 436/86

(57) ABSTRACT

Methods and products for identifying subjects at risk of acute kidney injury (AKI) are provided according to the invention. Included, for instance, are diagnostic kits and methods involving the use of at least two AKI associated markers.

### BACKGROUND OF INVENTION

Acute kidney injury (AKI) is a serious complication of cardiac surgery. Currently available tools for the preoperative risk stratification of acute kidney injury are imprecise. In the past, several agents have been used as potential treatments of AKI to impact the high mortality associated with AKI, without much success. One reason for the failure of these therapeutic interventions in clinical trials of AKI is the dependency on serum creatinine as a screening process for initial enrollment of patients, for the diagnosis of AKI and for initiating the intervention. The diagnosis of AKI based on a progressive rise in serum creatinine over several days can only be made with a significant delay and therefore also delays the treatment.

Several markers have been explored for early detection of AKI, including cytokines such as IL18 and other molecules such as kidney injury molecule-1 (KIM-1), cystein-rich protein 61 (Cry61), neutrophil gelatinase-associated lipocalin (NGAL) and sodium/hydrogen exchanger isoform 3 (NHE3). However, the use of biological markers as premorbid risk stratification tools for the development of AKI has not been explored.

### SUMMARY OF INVENTION

The invention in one embodiment is a method for identifying a subject having a risk of acute kidney injury by determining levels of at least two AKI associated markers in a subject, wherein a significant change in levels of the AKI associated markers relative to a standard level is indicative of a subject having a risk of acute kidney injury.

The subject may be a candidate for cardiac surgery with cardiopulmonary bypass. In this instance the results of the methods of the invention can be used to indicate that the subject should proceed with surgery, to indicate that the surgery should be delayed, or to institute prophylactic treatments. Methods for predicting candidates having a likelihood of suffering from AKI following cardiac surgery are imprecise and not widely used in clinical practice. The invention reduces the likelihood that a subject will have AKI by predicting the likelihood of occurrence of such a condition in advance of the surgery. The surgery can be postponed until the AKI associated markers described herein return to normal levels or prophylactic treatments can be instituted to reduce the risk for AKI.

In some embodiments the at least two AKI associated markers are selected from the group consisting of Myeloperoxidase (MPO), Plasminogen activator inhibitor 1 (PAI-1), monocyte inhibitory protein 1 (MIP-1α, MIP-1β), EGF, MCP-1, G-CSF, FRACT, IL-2, IL-6, IL-10, IL-12, TNFα, sICAM and soluble vascular cell adhesion molecule (sVCAM). In other embodiments the at least one or at least two AKI associated markers are selected from the group consisting of MPO, PAI-1, MIP-1β, EGF, MCP-1, G-CSF, FRACT, IL-2, IL-10, IL-12, TNFα, sICAM and sVCAM wherein an increase in the at least two AKI associated markers relative to the standard level is indicative of the subject having a risk of acute kidney injury. Optionally the levels of all of EGF, G-CSF, MIP-1β, and sVCAM are determined to identify a subject having a risk of acute kidney injury. In another embodiment the levels of all of MIP-1 and EGF are determined to identify a subject having a risk of acute kidney injury.

The levels of AKI associated markers are determined using protein isolated from the subject. The protein may be analyzed using a multiplex protein analyzer. In some embodiments the protein is detected in plasma isolated from the subject.

In still other aspects, levels of one or more AKI associated markers are mathematically combined with known clinical risk factors for AKI, readily available from an individuals prior and/or current medical condition.

A kit is provided according to other aspects of the invention. The kit may include at least two analytical reagents, wherein the analytical reagents are capable of binding to an AKI associated marker, housed in one or more containers, and instructions for identifying a subject having a risk of acute kidney injury by determining levels of at least two AKI associated markers, wherein a significant change in levels of the AKI associated markers relative to a standard level is indicative of a subject having a risk of acute kidney injury. In some embodiments the kit also includes one or more of a secondary reagent and a standard reagent, a label, a wash buffer or a container for carrying out a biomolecular reaction.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-D are a set of graphs depicting measurements of preoperative levels (in subjects scheduled to undergo cardiac surgery) of MPO; matrix metalloproteinase MMP-9; soluble adhesion molecules sE-selectin, sICAM, sVCAM; inflammatory cytokines TNF-α, TGF-α, IL-1Ra, IL-4, IL-6, IL-10, IL-12, IFN-γ; growth factors EGF, G-CSF, GM-CSF, VEGF; chemokines IL-8, MCP-1 MIP-1β, MIP-1 α, IP-10, fractalkine; and PAI-1.

### DETAILED DESCRIPTION

Improvements in the objective estimation of acute kidney injury (AKI) risk prior to cardiac surgery with cardiopulmonary bypass would allow at-risk patients to defer surgery or use preventive pharmacologic or other interventions including but not limited to antioxidants and would provide a more guided risk reduction strategy. The invention is based at least in part on the finding that the preoperative plasma concentration of AKI associated markers such as chemokines (including but not limited to Macrophage Inflammatory Protein 1β or MIP-1β (also known as CCL 4) and oxidative stress markers (including but not limited to myeloperoxidase or MPO) are associated with postoperative development of AKI. Examples of AKI associated markers include MPO, PAI-1, MIP-1α, MIP-1β, EGF, MCP-1, G-CSF, FRACT, IL-2, IL-6, IL-10, IL-12, TNFα, sICAM and sVCAM. Measurement of levels of AKI associated markers using conventional technology, such as with a multiplex protein immunologic assay technology, allows for the identification of at-risk individuals. The surgery date for these at-risk subjects may be delayed until an improvement in risk is documented by repeat measurements or allow for a guided use of preventive therapies or interventions prior to surgery. The methods of the invention should lead to a reduction in post cardiac surgery associated AKI. Thus, preoperative risk stratification for acute kidney injury in patients awaiting cardiac surgery; monitoring of acute kidney injury risk through serial measurements; preoperative risk stratification for other intra or postoperative complications such as systemic inflammatory response; prolonged mechanical ventilation or hemodynamic perturbations; and enhancement of clinical tools for preoperative acute kidney injury risk stratification through combined approaches are all uses of the invention.

AKI occurs when an injurious process damages the kidneys resulting in a rapid decline in the kidneys' ability to clear the blood of toxic substances. Temporarily, the kidneys cannot adequately remove fluids, electrolytes and wastes from the body or maintain the proper level of certain kidney-regulated chemicals leading to an accumulation of toxic metabolic waste products in the blood. AKI can result from any condition that decreases the blood supply to the kidneys, such as the conditions under which cardiopulmonary bypass is performed during cardiac surgery. Symptoms depend on the severity of kidney dysfunction, its rate of progression, and its underlying cause but can include anemia, fluid overload and edema, hypertension, fatigue, itching, lower back pain, nausea and vomiting, confusion and ultimately, coma and death. A decrease in glomerular filtration rate (GFR) is the principle functional change in patients with AKI.

In general, the methods of the invention include obtaining a tissue sample suspected of containing one or more of the AKI associated markers as a protein or RNA, and contacting the sample with an analytical reagent that is capable of binding to and identifying the presence of the AKI associated markers, under conditions effective to allow the formation of binding complexes. The levels of the AKI associated markers in the tissue sample can then be determined using routine methods known to those of skill in the art.

As used herein, a subject is a human, non-human primate, cow, horse, pig, sheep, goat, dog, cat, or rodent. In all embodiments human subjects are preferred. As used herein, a tissue sample is tissue obtained from a subject, preferably peripheral blood plasma, using methods well known to those of ordinary skill in the related medical arts.

The levels of AKI associated markers are compared to normal or baseline levels. A normal or baseline level may be determined based on known averages in tested populations of individuals or may be determined from a control sample run at the same time or in close proximity to the test sample. Those of skill in the art are very familiar with differentiating between significant expression of a biomarker, which represents a positive identification, and low level or background expression of a biomarker. Indeed, background expression levels are often used to form a “cut-off” above which increased staining will be scored as significant or positive. Significant expression may be represented by high levels of proteins within tissues or body fluids.

The methods may involve in some aspects detection of a protein found in peripheral blood plasma. Contacting the chosen biological sample with the protein under conditions effective and for a period of time sufficient to allow the formation of immune complexes (primary immune complexes) is generally a matter of simply adding the composition to the sample and incubating the mixture for a period of time long enough for the antibodies to form immune complexes with, i.e., to bind to, any antigens present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any radioactive, fluorescent, biological or enzymatic tags or labels of standard use in the art. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first added component that becomes bound within the primary immune complexes may be detected by means of a second binding ligand that has binding affinity for the encoded protein, peptide or corresponding antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the encoded protein, peptide or corresponding antibody is used to form secondary immune complexes, as described above. After washing, the secondary immune complexes are contacted with a third binding ligand or antibody that has binding affinity for the second antibody, again under conditions effective and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

Multiplexed assay methods may also be used in the invention. In some aspects the methods involve the use of Luminex assays. Luminex assays are based on xMAP technology (multi-analyte profiling beads) enabling the detection and quantitation of multiple RNA or protein targets simultaneously. The xMAP system combines a flow cytometer, fluorescent-dyed microspheres (beads), lasers and digital signal processing to effectively allow multiplexing of up to 100 unique assays within a single sample. The Luminex™ platform combines the efficiencies of multiplexing up to 100 different extracellular or intracellular markers for simultaneous analysis, with similar reproducibility to ELISA methodology. The BioSource Mercator Glass Slide Array is a pre-coated glass slide, which utilizes a patented technology for coating proteins. These slides allow for simultaneous multiplexing of phosphoproteins with minimal use of sample and an easy to use format. This product provides a broad (2-4 log) dynamic range, high sensitivity and exquisite reproducibility. The use of in-house manufactured, highly specific phospho- and pan antibodies as well as recombinant protein standards, allows the generation of accurate and quantitative measurement.

The mixture of the foregoing assay materials is incubated under conditions whereby, the AKI associated marker specifically binds the binding or analytical reagent. The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 0.1 and 10 hours.

After incubation, the presence or absence of specific binding between the AKI associated marker and one or more binding targets is detected by any convenient method available to the user. For cell-free binding type assays, a separation step is often used to separate bound from unbound components. The separation step may be accomplished in a variety of ways. Conveniently, at least one of the components is immobilized on a solid substrate, from which the unbound components may be easily separated. The solid substrate can be made of a wide variety of materials and in a wide variety of shapes, e.g., microtiter plate, microbead, dipstick, resin particle, etc. The substrate preferably is chosen to maximize signal-to-noise ratios, primarily to minimize background binding, as well as for ease of separation and cost.

Separation may be effected for example, by removing a bead or dipstick from a reservoir, emptying or diluting a reservoir such as a microtiter plate well, rinsing a bead, particle, chromotographic column or filter with a wash solution or solvent. The separation step preferably includes multiple rinses or washes. For example, when the solid substrate is a microtiter plate, the wells may be washed several times with a washing solution, which typically includes those components of the incubation mixture that do not participate in specific bindings such as salts, buffer, detergent, non-specific protein, etc. Where the solid substrate is a magnetic bead, the beads may be washed one or more times with a washing solution and isolated using a magnet.

Detection may be effected in any convenient way. For cell-free binding assays, one of the components usually comprises, or is coupled to, a detectable label. A wide variety of labels can be used, such as those that provide direct detection (e.g., radioactivity, luminescence, optical, or electron density, etc) or indirect detection (e.g., epitope tag such as the FLAG epitope, enzyme tag such as horseseradish peroxidase, etc.). The label may be bound to the analytical reagent, or bound to or incorporated into the structure of the secondary reagent.

A variety of methods may be used to detect the label, depending on the nature of the label and other assay components. For example, the label may be detected while bound to the solid substrate or subsequent to separation from the solid substrate. Labels may be directly detected through optical or electron density, radioactive emissions, nonradiative energy transfers, etc. or indirectly detected with antibody conjugates, strepavidin-biotin conjugates, etc. Methods for detecting the labels are well known in the art.

It should be appreciated that in one embodiment, aspects of the invention include methods wherein marker level data is sent to a remote site for analysis and an output is received from the remote site (e.g., comparison results and/or resulting recommendations, etc.). Similarly, in another embodiment, a marker level data may be received from a remote site, analyzed, and an output may be returned to the remote site.

Accordingly, aspects of the invention include methods wherein a patient recommendation may be made using existing data that may be received and/or be analyzed without performing a new assay.

In addition, aspects of the invention may include combining one or more marker level results as above described with clinical data that has previously been shown to be associated with individual risk for AKI. Clinical information that may be associated with AKI risk includes but is not limited to female gender, history of congestive heart failure, diabetes mellitus and/or chronic obstructive pulmonary disease, decreased left ventricular ejection fraction, preoperative use of intra-aortic balloon counterpulsation, history of previous cardiac surgery, emergency surgery, surgery type such as inclusion of heart valve procedures and preoperative baseline kidney function (Thakar et al. J Am Soc. Nephrol 16: 162-168, 2005). Aspects of the invention relate to providing threshold levels that are suitable for clinical algorithms described herein. Aspects of the invention also relate to business methods that may involve the marketing and/or licensing of techniques (e.g., clinical techniques, analytical techniques) and/or threshold levels described herein, including computer implemented methods for performing aspects of these techniques and/or electronic storage media containing sufficient information for use in one or more acts described herein. In one embodiment, one or more threshold levels or methods of using the threshold levels may be marketed to medical or research customers or potential customers. In one embodiment, a fee-based service may be provided to medical or research organizations wherein information relating to a marker, threshold level, or associated analysis or clinical algorithm may provided in exchange for a fee. The amount of the fee may be determined, at least in part, by the type of information that is provided, the type and degree of analysis that is requested, and the format and timing of the analysis.

It should be understood that aspects of the invention may be applicable to any suitable marker information and/or clinical algorithms. The sample or information may be received from many different sources, including, but not limited to one or more of the following: medical centers, large pharmaceutical companies (e.g., in association with pre-clinical evaluations or during clinical trials), CROs (for both pre-clinical and clinical analyses), medical laboratories and practices (e.g., scanning centers), hospitals, clinics, medical centers, small biotechnology companies (e.g., in association with pre-clinical evaluations or during clinical trials), and bio-medical research organizations. The results of the assays and/or analyses then may be returned to any one of these organizations. In some embodiments, the assay and/or analysis results may be returned to the same entity that sent the sample. In other embodiments, the results may be returned to a different entity. One or more steps involved with receiving the sample and/or data, assaying the sample and/or analyzing data, processing the results and forwarding the results to a recipient may be automated. It also should be appreciated that one or more of these steps may be performed outside the United States of America. Business procedures (e.g., marketing, selling, licensing) may be performed individually or collaboratively.

It should be appreciated that some or all of the diagnostic aspects of the invention can be automated as described herein.

Aspects of the invention may be implemented to follow up after and/or evaluate the effectiveness of a therapeutic intervention (e.g., a surgery or other therapeutic procedure).

It should be appreciated that some or all of the interventional aspects of the invention can be automated as described herein.

Aspects of the invention also can be used to optimize a therapeutic treatment for a patient. The disease status can be monitored in response to different treatment types or dosages, and an optimal treatment can be identified. The optimal treatment may change as the disease progresses. The effectiveness of the treatment over time can be monitored by analyzing changes in disease-associated levels of markers using the aspects of the present invention described herein. It should be appreciated that some or all of the therapeutic aspects of the invention can be automated as described herein.

The invention also includes articles, which refers to any one or collection of components. In some embodiments the articles are kits. The articles include compounds described herein in one or more containers. The article may include instructions or labels promoting or describing the use of the compounds of the invention.

As used herein, “promoted” includes all methods of doing business including methods of education, hospital and other clinical instruction, pharmaceutical industry activity including diagnostics, pharmaceutical sales, and any advertising or other promotional activity including written, oral and electronic communication of any form, associated with compounds described herein.

“Instructions” can define a component of promotion, and typically involve written instructions on or associated with packaging of compositions of the invention. Instructions also can include any oral or electronic instructions provided in any manner.

A “kit” typically defines a package including any one or a combination of the compounds described herein, including analytical reagents and the instructions, or homologs, analogs, derivatives, and functionally equivalent compositions thereof, but can also include a composition of the invention and instructions of any form that are provided in connection with the composition in a manner such that a clinical professional will clearly recognize that the instructions are to be associated with the specific composition.

The analytical reagents used herein are compounds that bind to one or more of the AKI associated markers and are useful in the diagnostic assays described herein.

The articles described herein may also contain one or more containers, which can contain compounds such as the compounds as described. The articles also may contain instructions for mixing, diluting, and/or administrating the compounds. The articles also can include other containers with one or more solvents, surfactants, preservative and/or diluents (e.g., normal saline (0.9% NaCl), or 5% dextrose) as well as containers for mixing, diluting or administering the components to the sample or to the patient in need of such treatment.

The compositions of the articles may be provided as any suitable form, for example, as liquid solutions or as dried powders. When the composition provided is a dry powder, the powder may be reconstituted by the addition of a suitable solvent, which may also be provided. In embodiments where liquid forms of the composition are used, the liquid form may be concentrated or ready to use.

The kits can include signal ligands for use with sandwich or competitive immunoassays. A signal ligand refers to a reactant, which is unassociated to any bead, capable of binding a target and being detected. A signal ligand can be, for example, any substance having associated therewith a detectable label such as a fluorescently- or radioactively-tagged antibody or antigen. The kit can also contain a binding partner for the signal ligand, which forms a complex with for example, an antibody, antigen, biotin, hapten, or analyte. The kits can include sets of particles for use as internal standards. Or else the kits can includes a set or sets of particles for use as controls. Or else the kits can include sets of particles for use as internal standards and a set or sets of particles for use as controls.

The articles, in one set of embodiments, may comprise a carrier means being compartmentalized to receive in close confinement one or more container means such as vials, tubes, and the like, each of the container means comprising one of the separate elements to be used in the method. For example, one of the container means may comprise a positive control in the assay. Additionally, the kit may include containers for other components, for example, buffers useful in the assay.

### EXAMPLES

### Example 1

### Multiplex Analysis of Plasma Proteins in Acute Kidney Injury Prior to and Following Cardiopulmonary Bypass

The pathophysiology of human acute kidney injury (AKI) following cardiopulmonary bypass (CPB) is poorly understood. The following study was performed to determine the plasma profile of 27 potential biomarkers in patients undergoing CPB by using a high-throughput multiplex system.

Methods: This was a nested case-control study (10 AKI and 10 control subjects) conducted within a large ongoing two-center prospective cohort study of cardiac surgery with CPB. Matching variables included sex, age, pre-operative left ventricular function, CPB time and surgery type. Plasma samples were obtained pre- and 2, 24 and 48 hours post CPB. AKI was defined as an increase in serum creatinine by 50% within the first 3 days of CPB. Using a multiplex protein analyzer (Luminex, Austin, Tex., USA), we measured levels of oxidative stress mediator MPO; matrix metalloproteinase MMP-9; soluble adhesion molecules sE-selectin, sICAM, sVCAM; inflammatory cytokines TNF-α, IL-1 α, IL-1 β, IL-1Ra, IL-2, IL-4, IL-6, IL-10, IL-12, IFN-α; growth factors EGF, G-CSF, GM-CSF, TGF-β, VEGF; chemokines IL-8, MCP-1 MIP-1 β, MIP-1 α, IP-10, fractalkine; and PAI-1.

Results: Plasma levels of MPO, MMP-9, sE-selectin, sVCAM, IL-1Ra, IL-6, IL-8, IL-10, G-CSF, MCP-1 and PAI-1 significantly increased in response to CPB (p<0.05 by Kruskal-Wallis). However, only IL-10, G-CSF and MIP-1 β were significantly higher in the AKI group compared to the control group (p<0.05 by Wilcoxon). MIP-1 α, MCP-1 and IL-2 were also higher but did not reach statistical significance (0.05p<0.10) whereas sE-selectin demonstrated a trend towards lower levels in the AKI group compared to controls (p=0.05).

Conclusion: Although several inflammatory-, leukocyte-, and oxidative stress markers, as well as growth factors are affected by CPB, only a few are strongly associated with AKI preceding and following CPB, and therefore are considered to be of higher importance in the pathophysiology or for the prediction of this syndrome.

### Example 2

### Preoperative AKI Risk Stratification in Cardiac Surgery

Preoperative tools for AKI risk stratification of individuals preparing for cardiac surgery are imprecise. The study was performed to evaluate the predictive value of plasma protein levels obtained preoperatively for the estimation of AKI risk in patients undergoing cardiac surgery.

Methods: A nested case-control study (10 AKI and 10 control subjects) was conducted within a large ongoing two-center prospective cohort study of cardiac surgery with CPB. Matching variables included sex, age, pre-operative left ventricular function, CPB time and surgery type. Plasma samples were obtained preoperatively. AKI was defined as an increase in serum creatinine by 50% within the first 3 days of cardiac surgery. Using a multiplex protein analyzer (Luminex, Austin, Tex., USA), levels of oxidative stress mediator MPO; matrix metalloproteinase MMP-9; soluble adhesion molecules sE-selectin, sICAM, sVCAM; inflammatory cytokines TNF-α, IL-1 α, IL-1 β, IL-1Ra, IL-2, IL-4, IL-6, IL-10, IL-12, IFN-α; growth factors EGF, G-CSF, GM-CSF, TGF-β, VEGF; chemokines IL-8, MCP-1 MIP-1β, MIP-1 α, IP-10, fractalkine; and PAI-1 were measured.

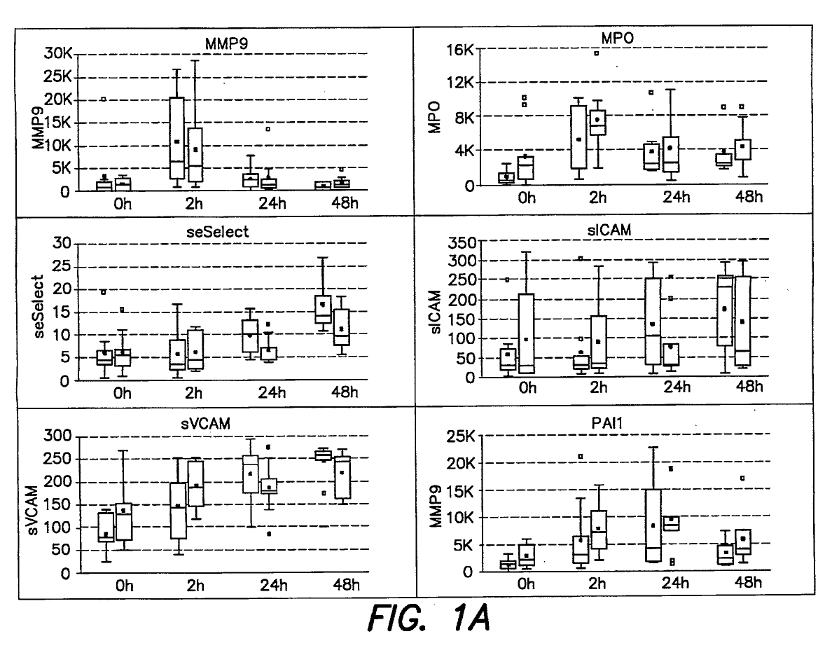

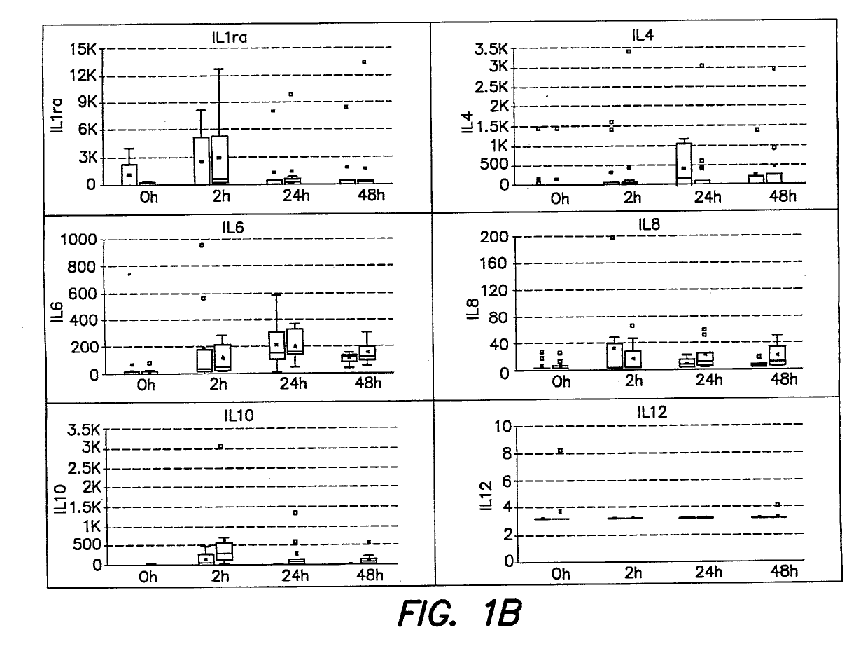

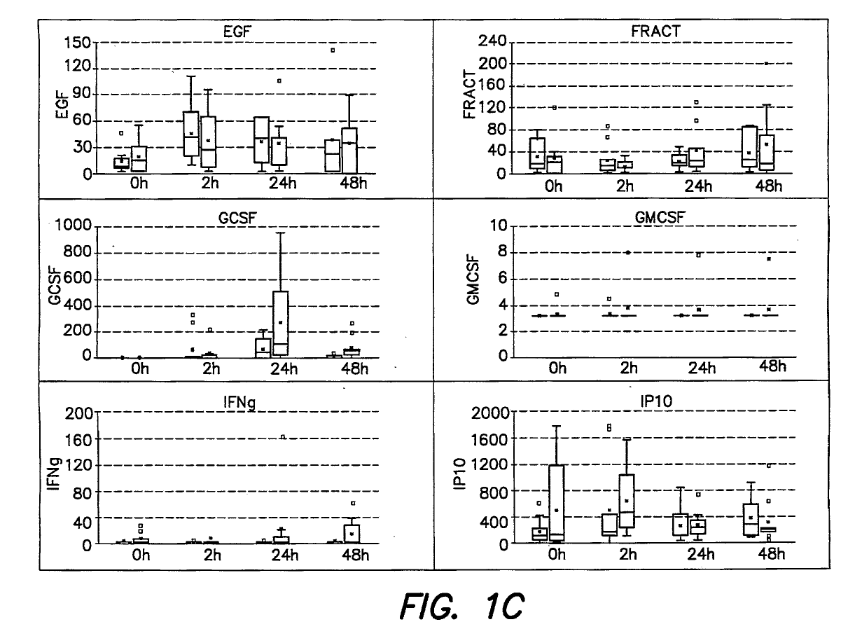

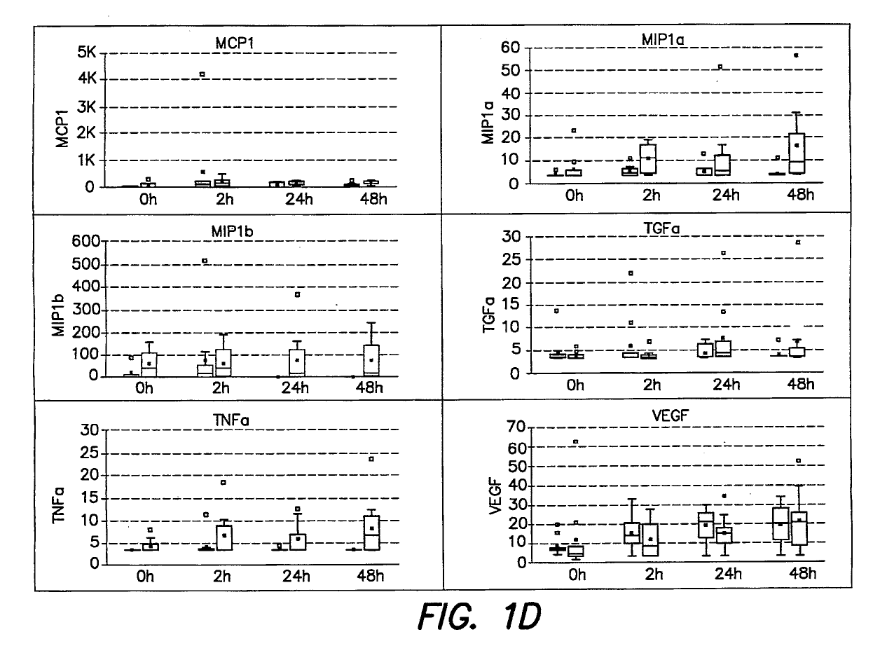

Results: Preoperative plasma levels of MPO, PAI-1, MIP-1, and TNF α were higher in patients destined to develop AKI following cardiac surgery. The area under the ROC curve for the prediction of AKI using preoperative MPO plasma levels was 0.77 (P=0.05). The data is shown in the form of box plots in FIG. 1.

Conclusion: Preoperative levels of plasma proteins may predict the risk of an individual for postoperative AKI and is thus useful as a method for preoperative AKI risk stratification.

### Example 3

### Plasma Protein Biomarker Patterns and Acute Kidney Injury Following Cardiopulmonary Bypass: A Nested Case Control Study

Background. Cardiac surgery that employs the technique of cardiopulmonary bypass (CPB) is a common procedure frequently associated with acute kidney injury, which represents an important complication and has detrimental effects in patient outcomes due to its strong association with morbidity and mortality. Despite this significance, little is known about the pathophysiology of acute kidney injury in this setting. Furthermore the ability to predict an individual patient's risk for AKI following cardiac surgery with CPB is limited.

Study Design. We created a nested case control study of 36 patients taken form a prospective cohort of adults undergoing on-pump cardiac surgery. 11 patients has AKI and 25 patient had no AKI. Matching variables included sex, age, pre-operative left ventricular function, CPB time and surgery type. Plasma samples were obtained pre- and 2, 24 and 48 hours post CPB.

Setting and Participants. Adult patients undergoing cardiac surgery in two tertiary, Boston-area medical centers.

Predictor. Peri-operative plasma concentrations of 27 biomarkers linked to a variety of pathophysiologic processes such as oxidative stress, inflammation, cell growth and differentiation, chemotaxis and cell adhesion. Using a multiplex protein analyzer (Luminex, Austin, Tex., USA), we measured levels of oxidative stress mediator MPO; matrix metalloproteinase MMP-9; adhesion molecules sE-selectin, sICAM, sVCAM; inflammatory cytokines TNF-α, IL-1α, IL-1β, IL-1Ra, IL-2, IL-4, IL-6, IL-10, IL-12, IFN-γ; growth factors EGF, G-CSF, GM-CSF, TGF-α, VEGF; chemokines IL-8, MCP-1 MIP-1α, MIP-1β, IP-10, fractalkine; and PAI-1.

Outcome. AKI, defined as a minimum 50% rise in serum creatinine within the first 72 hours.

Measurements. Plasma proteins before, and 2, 24, and 48 hours following CPB by Luminex. Area-under-the-receiver-operator characteristic curves (AUCs) were generated using a C statistic, and univariate logistic regression analyses were performed for the prediction of AKI.

Results. The characteristics of the nested case and control groups are shown in Table 1. It is evident that the two groups are similar with respect to age, gender distribution and several key preoperative demographic as well as intraoperative technical data, such as cardiopulmonary bypass time. While most of the biomarkers did not show differences in plasma concentration at any time point, some developed either higher or lower levels (shown in Table 2) at various time points. At the preoperative time point, EGF and MIP-1 beta were higher, whereas GCSF showed a trend toward lower plasma levels. EGF and MIP-1 beta and predicted AKI moderately well as indicated by an ROC area greater then 0.7 (table 2). At the time point 2 hours following discontinuation of CPB, MPO and sVCAM trended higher, whereas GCSF again was lower in the AKI compared to the non AKI group. The remainder of the results are summarized in tables 2 and 3.

Conclusion. This preliminary, broad evaluation of several perioperative plasma biomarkers allowed for the identification of a small number of biomarker candidates that demonstrate significant differences in perioperative plasma levels in patients undergoing cardiac surgery with CPB who developed acute kidney injury (AKI) compared with patients who did not develop AKI. The biomarkers MIP-1 beta, sVCAM and sICAM were most consistently different throughout the observation time points and therefore may be associated with the pathophysiology of AKI. MIP-1 beta and EGF showed differences between the AKI and non AKI groups already preoperatively, which might suggest their utility as preoperative prognostic stratification markers for AKI risk in patients planning to undergo cardiac surgery with CPB.

TABLE 1
Clinical characteristics of the study cohort

| | No AKI | AKI | Total Subjects | |
|---|---|---|---|---|
| | (N = 25, 69.4%) | (N = 11, 30.6%) | (N = 36) | P-value |
| Age | 70.4 ± 11.8 | 73.8 ± 8.6 | 71.5 ± 10.9 | 0.4015 |
| Gender (female) | 24.0% | 27.3% | 25.0% | 0.8345 |
| DM | 16.7% | 27.3% | 20.0% | 0.4665 |
| HTN | 70.8% | 72.7% | 71.4% | 0.9083 |
| H/o stroke | 4.2% | 18.2% | 8.6% | 0.1691 |
| Peripheral vasc disease | 12.5% | 27.3% | 17.1% | 0.2817 |
| Previous cardiovascular | 20.8% | 9.1% | 17.1% | 0.7725 |
| surgery | | | | |
| Without CPB | 4.2% | 0.0% | 2.9% | |
| Procedure status | | | | |
| Elective | 29.2% | 36.4% | 31.4% | 0.7433 |
| Urgent | 66.7% | 63.6% | 65.7% | |
| Emergent | 4.2% | 0.0% | 2.9% | |
| Valvular surgery | 66.7% | 90.9% | 74.3% | 0.3622 |
| Total duration of ICU stay | 3.2 ± 1.6 | 9.4 ± 12.4 | 5.1 ± 7.4 | 0.0185 |
| Ejection fraction | 52.7 ± 13.8 | 47.7 ± 17.1 | 51.1 ± 14.9 | 0.3649 |
| CPB time | 126.8 ± 36.5 | 142.5 ± 53.3 | 131.6 ± 42.1 | 0.3086 |
| Total time ventilated postop | 12.5 ± 5.5 | 114.9 ± 201.2 | 42.6 ± 115.4 | 0.0159 |
| Creatinine 24 h before | 1.1 ± 0.3 | 1.0 ± 0.2 | 1.1 ± 0.3 | 0.5613 |
| surgery | | | | |
| Creatinine 24 hours after | 1.1 ± 0.2 | 1.5 ± 0.3 | 1.2 ± 0.3 | 0.0001 |
| stop CPB | | | | |
| Creatinine 48 hours after | 1.2 ± 0.4 | 1.8 ± 0.5 | 1.4 ± 0.5 | 0.0005 |
| stop CPB | | | | |
| Creatinine 72 hours after | 1.2 ± 0.5 | 1.8 ± 0.7 | 1.4 ± 0.6 | 0.0024 |
| stop CPB | | | | |

Mean ± sd or %; P by chi square or t-test

TABLE 2
Association of selected plasma biomarker levels
with the development of post-CPB AKI.

| | | |
|---|---|---|
| No AKI | AKI | |
| (N = 25, 69.4%) | (N = 11, 30.6%) | P value |

Pre CPB time point

| | | | |
|---|---|---|---|
| egf_0h | 9.8 ± 12.6 | 19.4 ± 16.4 | 0.0439 |
| gcsf_0h | 23.5 ± 61.0 | 3.2 ± 0.0 | 0.0684 |
| mip1b_0h | 17.7 ± 31.1 | 54.9 ± 56.1 | 0.0157 |

2 hours post CPB

| | | | |
|---|---|---|---|
| mpo_2h | 6540.8 ± 12039.0 | 7009.8 ± 3868.5 | 0.0858 |
| svcam_2h | 134.3 ± 69.8 | 184.8 ± 53.6 | 0.0440 |
| fract_2h | 12.0 ± 21.0 | 14.3 ± 10.2 | 0.0358 |
| gcsf_2h | 213.8 ± 291.3 | 50.5 ± 83.8 | 0.0950 |
| mip1a_2h | 12.3 ± 38.5 | 10.0 ± 6.2 | 0.0464 |

24 hours post CPB

| | | | |
|---|---|---|---|
| mip1b_24h | 29.7 ± 85.9 | 70.7 ± 116.8 | 0.0751 |

48 hour post CPB time point

| | | | |
|---|---|---|---|
| svcam_48h | 268.9 ± 94.4 | 206.6 ± 55.9 | 0.0346 |
| il6_48h | 278.5 ± 232.5 | 142.9 ± 81.8 | 0.0833 |
| il12_48h | 16.1 ± 24.7 | 3.3 ± 0.3 | 0.0443 |

Logistic regression models were generated to indicate odds ratios per doubling in plasma bionmarkers. OR denotes odds ratio; CI confidence interval; and CPB, cardiopulmonary bypass.
mean ± sd; P value by Wilcoxon Rank Sum test

TABLE 3
Area under the ROC curve (AUC) of selected
variables for the prediction of AKI.

| | | | |
|---|---|---|---|
| OR (95% CI) | P value | ROC | P value |

Pre CPB time point

| | | | | |
|---|---|---|---|---|
| egf_0h_log | 2.23 (1.01, 4.90) | 0.047 | 0.71 | 0.038 |
| mip1b_0h_log | 1.83 (1.07, 3.12) | 0.026 | 0.74 | 0.020 |

2-hour post CPB time point

| | | | | |
|---|---|---|---|---|
| svcam_2h_log | 7.17 (0.92, 55.80) | 0.060 | 0.73 | 0.028 |

24-hour post CPB time point

| | | | | |
|---|---|---|---|---|
| sicam_24h_log | 0.50 (0.23, 1.09) | 0.082 | 0.68 | 0.070 |

48-hour post CPB time point

| | | | | |
|---|---|---|---|---|
| sicam_48h_log | 0.46 (0.20, 1.06) | 0.067 | 0.66 | 0.051 |
| svcam_48h_log | 0.04 (0.00, 1.03) | 0.052 | 0.74 | 0.026 |
| il6_48h_log | 0.35 (0.10, 1.16) | 0.085 | 0.70 | 0.060 |
| il12_48h_log | 0.07 (0.00, 19.76) | 0.349 | 0.69 | 0.010 |

ROC denotes receiver operator characteristic; CI, confidence interval; and CPB, cardiopulmonary bypass.

### Example 4

### Statistical Determination of the Cut-Off Values of Preoperative Plasma Levels of MIP-1 and EGF, Above which the Risk of Acute Kidney Injury Increases

Methods: Test performance characteristics for the early detection of AKI were evaluated for each biomarker using a receiver operator characteristic curve (ROC) analysis. In addition, biomarkers with the best area-under-the-ROC curve (AUC) were combined, and biomarker panels with or without the addition of CPB perfusion time were evaluated with the same method. 95% confidence interval for the AUC was calculated, and formal statistical testing was performed using the non-parametric method of DeLong (DeLong E R, DeLong D M, Clarke-Pearson D L. Comparing the areas under JO two or more correlated receiver operating characteristic curves: a nonparametric approach. Biometrics 44 (3): 837-845, 1988).

Optimal cut-off points for early detection of AKI were determined for each biomarker using the Youden index (Youden W J. Index for rating diagnostic tests. Cancer 3 (1): 32-35, 1950), based on which sensitivity, specificity, positive and negative predictive values were calculated.

Results: MIP-1: An increase in preoperative plasma MIP-1 beta levels is associated with a 1.83 fold higher odds ((95% CI 1.07-3.12; P=0.026) of AKI, per each doubling of MIP-1 beta level. The ROC area under the curve is estimated at 0.74 (P=0.038) and values over 28.82 pg/ml detect AKI with a sensitivity of 0.64 and specificity of 0.82.

EGF: An increase in preoperative plasma EGF levels is associated with a 2.23 fold higher odds (95% CI 1.01-4.90; P=0.047) of AKI, per each doubling of EGF level. The ROC are under the curve is estimated at 0.71 (P=0.038) and values over 7.61 pg/ml detect AKI with a sensitivity of 0.72 and specificity of 0.69.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A method for identifying a subject having a risk of acute kidney injury (AKI), comprising:
determining levels of at least two AKI associated markers in a subject, wherein a significant change in levels of the AKI associated markers relative to a standard level is indicative of a subject having a risk of acute kidney injury.

2. The method of claim 1, wherein the at least two AKI associated markers are selected from the group consisting of MPO, PAI-1, MIP-1α, MIP-1β, EGF, MCP-1, G-CSF, FRACT, IL-2, IL-6, IL-10, IL-12, TNFα, sICAM and sVCAM.

3. The method of claim 1, wherein the at least two AKI associated markers are selected from the group consisting of MPO, PAI-1, MIP-1α, MIP-1β, EGF, MCP-1, G-CSF, FRACT, IL-2, IL-6, IL-10, IL-12, TNFα, sICAM and sVCAM, wherein an increase or decrease in the at least two AKI associated markers relative to the standard level is indicative of the subject having a risk of acute kidney injury.

4. The method of claim 1, wherein at least one AKI associated marker is selected from the group consisting of MPO, PAI-1, MIP-1α, MIP-1β, EGF, MCP-1, G-CSF, FRACT, IL-2, IL-6, IL-10, IL-12, TNFα, sICAM and sVCAM, wherein an increase or decrease in the AKI associated markers relative to the standard level is indicative of the subject having a risk of acute kidney injury.

5. The method of claim 1, wherein the subject is a candidate for cardiac surgery with cardiopulmonary bypass.

6. The method of claim 1, wherein levels of all EGF, G-CSF, MIP-1β, and sVCAM are determined to identify a subject having a risk of acute kidney injury.

7. The method of claim 1, wherein levels of all MIP-1 and EGF are determined to identify a subject having a risk of acute kidney injury.

8. The method of claim 1, wherein levels of AKI associated markers are determined using protein isolated from the subject.

9. The method of claim 8, wherein the protein is analyzed using a multiplex protein analyzer.

10. The method of claim 9, wherein the protein is detected in plasma isolated from the subject.

11. A kit, comprising
at least two analytical reagents, wherein the analytical reagents are capable of binding to an AKI associated marker, housed in one or more containers, and
instructions for identifying a subject having a risk of acute kidney injury by determining levels of at least two AKI associated markers, wherein a significant change in levels of the AKI associated markers relative to a standard level is indicative of a subject having a risk of acute kidney injury.

12. The kit of claim 11, further comprising a secondary reagent and a standard reagent.

13. The kit of claim 11, further comprising a label.

14. The kit of claim 11, further comprising a wash buffer.

15. The kit of claim 11, further comprising a container for carrying out a bimolecular reaction.

16. The method of claim 1, wherein levels of one or more AKI associated markers are mathematically combined with known clinical risk factors for AKI, readily available from an individuals prior and/or current medical condition.
